(19) United States
(12) Patent Application Publication
(10) Pub. No.: US 2023/0038070 A1
(43) Pub. Date: Feb. 9, 2023

(54) POLYMER MATRIXES FOR DIFFERENT COMPOSITIONS OF MITOCHONDRIALLY TARGETED ANTIOXIDANTS

(71) Applicant: Miotech S.A., Luxembourg (LU)

(72) Inventors: Maxim SKULACHEV, Moscow (RU); Natalia MARMIY, Moscow (RU); Vadim TASHLITSKY, Moscow (RU); Lawrence FRIEDHOFF, River Vale, NJ (US)

(73) Assignee: MITOTECH S.A., Luxembourg (LU)

(21) Appl. No.: 17/783,743

(22) PCT Filed: Dec. 10, 2020
(86) PCT No.: PCT/US20/64334

§ 371 Date: Jun. 9, 2022

Related U.S. Application Data
(60) Provisional application No. 62/945,939, filed on Dec. 10, 2019.

(51) Int. Cl. A61K 31/66; A61K 47/12; A61K 47/10; A61K 47/32; A61K 8/55; A61K 8/02

(52) U.S. Cl. CPC A61K 31/66; A61K 47/12; A61K 47/10; A61K 47/32; A61K 8/55; A61K 8/0212

(57) ABSTRACT

A composition containing amitochodrially-targeted antioxidant of the SkQ type and a polyacrylate, agarose, agar-agar orhyaluronic acid polymer useful for production of pharmaceutical, medicinal and cosmetic compositions having good stability. Such compositions may usefully also include lactic acid.

### CROSS REFERENCE TO THE RELATED APPLICATION

This application claims the benefit to U.S. Provisional Patent Application No. 62/945,939, filed Dec. 10, 2019, the contents of which are incorporated herein by reference.

### SUMMARY OF THE INVENTION

This invention relates to pharmaceutics, medicine and cosmetics. The invention describes several stable compositions comprising a mitochondrially targeted antioxidant, a polymer matrix and optionally additional components.

### BACKGROUND OF THE INVENTION

Mitochondrially targeted antioxidants of SkQ type (SkQs, see general formula I) are not stable in compositions comprising many ingredients common in pharmaceutical or cosmetic compositions. This makes SkQs not compatible (in the view of stability required for some reasonable storage duration) with many typical ingredients of pharmaceuticals and\or cosmetics.

### DESCRIPTION OF THE INVENTION

This invention provides several reasonably stable compositions containing mitochondrially targeted antioxidants of SkQ type, wherein mitochondrially targeted antioxidants of SkQ type have general formula 1:

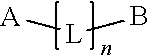

wherein:
A is a quinone antioxidant having a following structure:

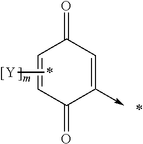

and/or reduced (quinole) form thereof, wherein:
   m is an integer from 1 to 3;
   Y is lower alkyl or lower alkoxy
L is a linker group, comprising straight or branched hydrocarbon chain which can be optionally substituted by one or more substituents and optionally contains one or more double or triple bonds; and
n is integer from 1 to 40;
B is a targeting group comprising SkZ, wherein Sk is a lipophilic cation; and Z is a pharmacologically acceptable anion.

SkQ 1 is a non-limiting example of SkQ type compounds (SkQs). The formula of SkQ1 (oxidized form) is:

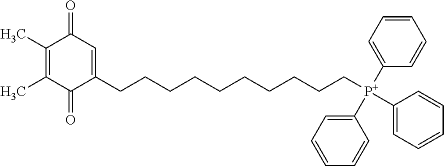 [SMILES: CC1=C(C)C(=O)C(CCCCCCCCCC[P+](c2ccccc2)(c2ccccc2)c2ccccc2)=CC1=O]

SkQ1 was used in the following experiments in a firm of bromide or chloride. In our experiments we demonstrated that many common components of pharmaceuticals and cosmetics are not compatible with SkQs because these components destabilize SkQs thus making the storage time of corresponding compositions short. However, we have found that the following polymers and other components do not destabilize significantly SkQs when combined with the mitochondrial targeted antioxidant in a composition (see table 1):

TABLE 1

| | | Reference in |
|---|---|---|
| # | Polymer or other component | this document |
| 1 | Carboxyvynil polymer (other names: | Exp. Example 1 |
| | carbomer, polyacrylate) | |
| 2 | Lactic acid (we have found that lactic acid is | Exp. Example 1 |
| | unexpectedly potent stabilizer of SkQs, i.e. SkQ1 | |
| | stability in a composition can be increased by | |
| | addition of lactic acid into the composition) | |
| 3 | pentyleneglycol, glycerol | |
| 4 | Agarose/Agar-agar | Exp. Example 2 |
| 5 | Polyvynil alcohol, Aquaxyl | Exp. Example 2 |
| 6 | Hyaluronic acid, glycolic acid, mandelic acid, | Exp. Example 2 |
| | lactobionic acid, aloe juice | |
| 7 | Algae extract Seafill, beta-glucan | Exp. Example 2 |

Several compositions provided high SkQ1 stability in our experiments (see experimental example 1). The compositions are:

Composition 1:

| | |
|---|---|
| Propyleneglycol | 3% |
| Pentyleneglycol | 5% |
| Sodium polyacrylate | 0.9% |
| Ethylhexylglycerol | 0.3% |
| Lactic acid | 0.2% |

SkQ1-different concentraions,
for example 6 ug/ml
Water-to 100%

Stability study is presented in the experimental example

Composition 2:

| | |
|---|---|
| Isopentyldiol | 5% |
| Aquaxyl (xylitylglacoside and | −3% |
| anhydroxylytol and xylitol) | |
| Propyleneglycol | 3% |
| Sodium polyacrylate | 0.9% |
| Ethylhexylglycerol | 0.3% |
| Lactic acid | 0.2% |

SkQ1-different concentrations,
for example 6 ug/ml
Water-to 100%

### Experimental Example 1. Polyacriclates as Suitable Polymer Matrixes for SkQs

### (A) The Study of Stability of Cosmetic Formulations on the Basis of Carbomer 640 and 641

Sample preparation: 1 V sample+1 V NaBr 0. 5 M, mix, 15 min on a shaker at 70 C,+9 V ethanol 96% fractional by 1 V, centrifuge. To 500 mcl of super add 500 mcl 0, 1 M of phosphoric acid in water, centrifuge and transfer to a vial. The final dilution of 22 times

Injection volume: 100 μl, detection at 260 nm.

| No | Description | Code | Composition |
|---|---|---|---|
| 1 | Carbomer 641 | C088-060819-03 | Propylene glycol-8% |
| | based hydrogel | | Ethylhexylglycerin-0.3% |
| | | | Carbomer 641-0.9% |
| | | | Lactic acid-0.2% |
| | | | SkQ1 50 uM |
| | | | water |
| | | | pH 5.5 |
| 2 | Carbomer 641 | C088-060819-04 | Propylene glycol-8% |
| | based hydrogel | | Ethylhexylglycerin-0.3% |
| | | | Carbomer 641-0.9% |
| | | | SkQ1 50 uM |
| | | | water |
| | | | pH 5.5 |
| 3 | Carbomer 641 | C088-060819-05 | Propylene glycol-8% |
| | based hydrogel | | Carbomer 641-0.9% |
| | | | Lactic acid-0.2% |
| | | | SkQ1 50 uM |
| | | | water |
| | | | pH 5.5 |
| 4 | Carbomer 640 | C088-060819-06 | Propylene glycol-8% |
| | based hydrogel | | Ethylhexylglycerin-0.3% |
| | | | Carbomer 640-0.9% |
| | | | Lactic acid-0.2% |
| | | | SkQ1 50 uM |
| | | | water |
| | | | pH 5.5 |

Results of analysis after +60° C. incubation

| Time at | | C., ug/g | | | |
|---|---|---|---|---|---|
| 60 C., | Sample date | C088- | C088- | C088- | C088- |
| days | collection | 060819-03-60 | 060819-04-60 | 060819-05-60 | 060819-06-60 |
| 0 | Aug. 8, 2019 | 40.28471 | 28.01799 | 57.00258 | 58.10061 |
| 4 | Aug. 12, 2019 | 14.16035 | 0.92242 | 8.879284 | 24.65781 |
| 8 | Aug. 16, 2019 | 8.680195 | 0.62347 | 15.21795 | 19.59364 |
| 11 | Aug. 19, 2019 | 6.12771 | 0.03129 | 8.886293 | 12.3145 |
| 15 | Aug. 23, 2019 | 8.013269 | | 5.85823 | 9.427738 |
| 18 | Aug. 26, 2019 | 5.256037 | | 14.1804 | 3.436147 |
| 22 | Aug. 30, 2019 | 2.787277 | | 13.86665 | 2.92139 |
| 25 | Sep. 2, 2019 | 1.947372 | | 6.041945 | 1.049452 |
| 29 | Sep. 6, 2019 | 1.720692 | | 9.569733 | 0.467825 |
| 32 | Sep. 9, 2019 | 1.257592 | | 11.77894 | 0.410404 |

Compositions 03 and 05 demonstrated pronounced degradation (with biphasic kinetics).

| | | Initial | | | | | |
|---|---|---|---|---|---|---|---|
| | | concentration | | | | | |
| No of | −k′ d − 1 | of the linear | Degradation rate for 365 days of storage, % | | | | |
| composition | 60 C. | section, ug/g | 60 C. | 25 C. | 8 C. | 4 C. | 2 C. |
| C088-060819- | −0.08505 | 19.59137 | 100.0% | 93.1% | 56.1% | 46.4% | 41.9% |
| 03-60 | | | | | | | |
| C088-060819- | −0.55671 | 28.01799 | 100.0% | 100.0% | 99.5% | 98.3% | 97.1% |
| 04-60 | | | | | | | |
| C088-060819- | −6.82E−04 | 10.05812 | 21.6% | 2.1% | 0.7% | 0.5% | 0.4% |
| 05-60 | | | | | | | |
| C088-060819- | −0.1577 | 58.10061 | 100.0% | 99.3% | 78.3% | 68.6% | 63.5% |
| 06-60 | | | | | | | |

A certain stability is observed only for the composition C088-060819-05-60. provided the initial concentration is not more than 10 micrograms.

### The Study of the Stability of the New Composition of Plastic0 Package

Method of sample preparation: Place 200 mg of the test polyacrylate composition (exact weight) in Eppendorf test tube, add 2 volumes of water (about 400 μl), and heat for 30 minutes (on a thermoshaker at 70 C). Add 1 volume of NaBr 1 M in water (about 200 μl), heat while mixing for another 30 minutes (on a thermoshaker at 70 C). Add 2 volumes of 96% ethanol (about 400 μl), mix on a vortex, and centrifuge. Select 900 μl of the supernatant and add 300 μl of the phosphoric acid solution in ethanol, mix, and centrifuge.

Final dilution ratio: 8 times.

The injection volume is 100 μl.

Test polyacrylate composition:

| | |
|---|---|
| SkQ1 bromide | 6.2 μg |
| Propylene glycol | 0.03 ml |
| Pentyleneglycol | 0.05 ml |
| Sodium polyacrylate | 0.9 mg |
| Ethylhexylglycerol | 0.3 mg |
| Lactic acid | 0.2 mg |
| Water | up to 1 ml |

Accelerated study of stability was performed at +60° C. Samples were prepared as described above and analyzed for SkQ1 concentration by HPLC method (injected volume 100 ul, detection at 260 nm).

Analysis results:

| C., | time, d |
|---|---|
| ug/ml | 60 C. |
| 5.88 | 0 |
| 5.78 | 3 |
| 5.69 | 7 |
| 5.54 | 10 |
| 4.99 | 14 |
| 4.72 | 17 |
| 4.46 | 21 |
| 4.21 | 24 |
| 4.23 | 28 |
| 4.17 | 31 |

Extrapolation of kinetic constants to actual storage conditions

| | −k′ d − 1 | Degradation rate for 365 days of storage, % | | | | |
|---|---|---|---|---|---|---|
| | 60 C. | 60 C. | 25 C. | 8 C. | 4 C. | 2 C. |
| Test | −1.30E−02 | 99.0% | 33.7% | 11.9% | 9.1% | 8.0% |
| polyacrylate | | | | | | |
| composition | | | | | | |

Conclusion: SkQ1 in the polyacrylate composition demonstrate acceptable stability.

(B) Stability study of carbomer-based gels 980 (10 uM and 50 mM SkQ1 concentration) in the presence of glycerol.

| No | Manic | Code | Composition |
|---|---|---|---|
| 1 | Pharmaceutical | C088- | SkQ1 (50 uM), lactic acid, sodium lactate, glycerol |
| | composition for external | 230119-01 | 1%, carbomer 980-, benzalkonium chloride 0.01% |
| | use based on carbomer 980 | (50 uM) | |
| 2 | Pharmaceutical | C088- | SkQ1 (10 uM), lactic acid, sodium lactate, glycerol |
| | composition for external | 230119-02 | 1%, carbomer 980-, benzalkonium chloride 0.01% |
| | use based on carbomer 980 | (10 uM) | |

Stability curves at +60° C.:

Kinetics of SkQ1 degradation at 60 C in the composition of carbomer 980-based gels.

Extrapolation of the average rates at actual storage conditions.

| No of | −k′ d − 1 | Degradation rate for 365 days of storage, % | | | | |
|---|---|---|---|---|---|---|
| composition | 60 C. | 60 C. | 25 C. | 8 C. | 4 C. | 2 C. |
| C088- | −0.02127 | 99.9% | 48.8% | 18.6% | 14.5% | 12.7% |
| 230119-02 | | | | | | |
| (10 uM) | | | | | | |
| C088- | −0.03519 | 100.0% | 67.0% | 28.9% | 22.8% | 20.1% |
| 230119-01 | | | | | | |
| (50 uM) | | | | | | |

Conclusion: The rate of degradation slightly depends on the initial concentration, which indicates the absence of an autocatalytic degradation mechanism. The required stability is not observed.

In the next experiment, we investigated the stability of SkQ1 in compositions at elevated temperatures. Together with the control compositions, the following composition (code MitoVitan—1-3) was studied:
Isopentyldiol 5%
Aquaxyl (xylitylglucoside and anhydroxylytol and xylitol)—3%
Propylenegycol 3%
Sodium polyacrylate 0.9%
Ethylhexylglycerol 0.3%
Lactic acid 0.2%
SkQ1—6 ug/ml
Water up to 100%.

Extrapolation of kinetic constants to actual storage conditions:

| | −k′ d − 1 | Degradation rate for 365 days of storage, % | | | | |
|---|---|---|---|---|---|---|
| | 60 C. | 60 C. | 25 C. | 8 C. | 4 C. | 2 C. |
| MitoVitan-1- | −0.02233 | 100.0% | 50.5% | 19.4% | 15.1% | 13.3% |
| 3 | | | | | | |

The MitoVitan-1-3 composition demonstrates acceptable stability in a model experiment of accelerated storage (at elevated temperature). Further experiments at +37 C and room temperature showed greater stability of SkQ in this composition compared to the calculated one.

Additional conclusions from experiments above:

Comparison of the stability of compositions C088-060819-03 and C088-060819-04 demonstrates that addition of lactic acid stabilizes SkQ1 . These experiments also revealed an unexpected destabilizing effect of surfactants (ethylhexylglycerol) present in the composition of C088-060819-05 and having dramatically worse stability compared to the same composition without ethylhexylglycerol (C0884)60819-03).

Based on the results of our experiments, we can conclude that various polyacrylates are well compatible with SkQ1 and can serve as a polymer base for cosmetic and pharmaceutical compositions of mitochondrial-targeted antioxidants. At the same time, the best compatibility (stability of SkQ1) was demonstrated by carbomers 640, 641 and 974. Unexpectedly, carbomer 980 was worse than the above.

The stabilizing effect of pentylene glycol and glycerol was also revealed.

### Experimental Example 2. Agar-Agar and Agarose as Suitable Natural Polymer Matrixes for SkQs

(A) Compositions Based on Agarose and Agar-Agar. Stability Study at 60 C

### Composition:

Agar (or agarose)—5%,
Lactic acid (sodium lactate)—1 %,
propylene glycol—20%.
SkQ1—50 microns.
Water
   Sample preparation (sample weight 300-500 mg):

From 300 to 500 mg of the composition (exact weight) was placed in a test tube after incubation at +60° C. temperature. Collected sample was melted on a thermoshaker at 70 C for 10 minutes and a volume of about 300-500 μl of acetonitrile is added (a volume is equal to the exact weight). The mixture was thoroughly mixed and centrifuged at 14000 rpm for 10 minutes, then 400 μl of the supernatant was transferred into a 1.5 ml tube, 600 μl of acetonitrile was added, mixed and thoroughly centrifuged at 14000 rpm for 30 minutes. Finally 400 μl of the supernatant was transferred into a chromatographic vial, 600 μl of water was added and the mixture was thoroughly mixed. 3 samples were taken for each time point.

Measurement results in different time points:

| | | Time at |
|---|---|---|
| SkQ1 | StDev, | 60 C., |
| μg/g | μg/g | days |
| 306.4 | 116.2 | 0 |
| 171.5 | 37.0 | 3 |
| 129.2 | 1.0 | 6 |
| 103.7 | 6.2 | 9 |
| 87.4 | 6.7 | 13 |
| 82.2 | 3.8 | 16 |
| 86.9 | 1.9 | 20 |
| 89.5 | 7.2 | 23 |
| 63.0 | 1.4 | 27 |
| 77.7 | 0.5 | 30 |

Extrapolation of the initial section of the kinetic curve to actual storage conditions:

| | Temperature | | | | | | |
|---|---|---|---|---|---|---|---|
| No of | −k′ d − 1 | Degradation rate for 12 mes of storage, % | | | | | |
| composition | 60 C. | 60 C. | 37 C. | 25 C. | 8 C. | 4 C. | 2 C. |
| C088- | −0.10905 | 100 | 100 | 97 | 66 | 56 | 51 |
| 070918-02 | | | | | | | |

Conclusion: The initial phase of degradation is replaced by the second phase where concentration is constant The ointment is very likely not to have the necessary stability at 2-8 C storage. It is likely that for this type of polymer it is not fully correct to extrapolate the data obtained during storage at +60 C to significantly lower temperatures (25 C, 2-8 C) due to the features of the polymer used. It is necessary to conduct a long experiment at lower temperatures (see below).

(B) Compositions Based on Agarose. Storage at +25° C.

### Composition:

Agar (or agarose)—5%,
Lactic acid (sodium lactate)—1%,
propylene glycol—20%.
SkQ1—50 microns,
Water.

Sampling: 3 eppendorfs per point. 25 C 1 time a month.

Sample preparation (sample weight 300-500 mg):

The test tube contains from 300 to 500 mg of the composition (exact weight). After incubation at +25° C., the sample is melted on a thermoshaker at 70 C for 10 minutes and a volume of about 300-500 μl of acetonitrile is added (volume equal to the exact weight). Mixture is thoroughly mixed and centrifuged at 14000 rpm for 10 minutes. 400 μl of the supernatant is transferred into a 1.5 ml tube, 600 μl of acetonitrile is added, mixed and thoroughly centrifuged at 14000 rpm for 30 minutes. 400 μl of the supernatant is transferred into a chromatographic vial and 600 μl of water is added followed by mix. The final dilution is 12.5 times, HPLC injection volume of 20 μl, detection at 260 nm.

Results of measurement of selected samples:

| | | Time at |
|---|---|---|
| SkQ1 | StDev, | 60 C., |
| μg/g | μg/g | days |
| 251.5 | 39.9 | 0 |
| 250.5 | 30.2 | 1 |
| 224.2 | 8.0 | 2 |
| 264.0 | 22.9 | 3 |
| 260.4 | 16.3 | 4 |
| 242.8 | 39.7 | 5 |
| 267.2 | 62.7 | 6 |

Conclusion: Degradation during 6 months at 25 C was not detected. Similar results were obtained at 25° C. when agarose was replaced with agar-agar.

(B) Study of the stability of SkQ1 in multi-active cosmetic masks.

Summary table:

| | | | The initial |
|---|---|---|---|
| | | | real SkQ1 |
| | | | concentration |
| No | Name/Code | Composition | μg/g |
| 1 | Base neutral mask/ | SkQ1, agar-agar, lactic acid, sodium | 8.339 |
| | C088-140219-01 | lactate, sodium hyaluronate, glycerin | |
| 2 | Base acid mask/ | SkQ1, agar-agar, lactic acid, hyaluronic | 10.726 |
| | C088-140219-02 | acid, glycerin | |
| 3 | Full acid mask/ | SkQ1, agar-agar, lactic acid, hyaluronic | 16.168 |
| | C088-140219-03 | acid, glycerin, glycolic acid, mandelic | |
| | | acid, lactobionic acid, aloe juice | |
| 4 | Full neutral mask/ | SkQ1, agar-agar, lactic acid, sodium | 4.754 |
| | C088-140219-04 | lactate, sodium hyaluronate, glycerin. | |
| | | aloe juice, algae extract, tremella extract, | |
| | | xylitol, ectoin, beta-glucan. | |

Sample preparation for all masks: 1 V of the sample (about 200 mg)+1 V 0.5M of sodium bromide, heat for 15 minutes while mixing, take 100 μl of the resulting solution and add gradually 900 μl of 96% ethanol, mix after each addition of ethanol, centrifuge. Take 500 ml. of supernatant, add 500 ml of phosphoric acid in water, centrifuge, transfer to vials. The final dilution is 40 times and the HPLC injection volume is 100 μl. Detection at 260 nm.

### Stability at +60° C.

Degradation curves (kinetics of degradation. of SkQ1 at 60° C.):

Extrapolation of kinetic constants to actual storage conditions.

| No of | −k′ d − 1 | Degradation rate for 365 days of storage, % | | | | |
|---|---|---|---|---|---|---|
| composition | 60 C. | 60 C. | 25 C. | 8 C. | 4 C. | 2 C. |
| C088- | −0.07215 | 100.0% | 89.7% | 50.3% | 41.1% | 36.9% |
| 140219-01 | | | | | | |
| C088- | −0.0148 | 99.5% | 37.2% | 13.4% | 10.3% | 9.0% |
| 140219-02 | | | | | | |
| C088- | −0.01522 | 99.6% | 38.1% | 13.7% | 10.6% | 9.3% |
| 140219-03 | | | | | | |
| C088- | −0.02787 | 100.0% | 58.4% | 23.7% | 18.5% | 16.3% |
| 140219-04 | | | | | | |

Conclusion: Compositions No 2 and No 3 have the best stability.

Further experiments confirmed the stability of SkQ1 in compositions C088-140219-02 n C088-140219-03 when stored at +37 C (no sums of SkQ1 degradation were detected for 6 months).

The following compositions were also tested (with a pH close to neutral):

Composition C088-1604194)7:

SkQ1-50 μM
agar-agar 0.25%
Sodium lactate-5%
Glycerin-10%
aloe juice-4%
algae extract (Seafill)-2%
beta-glucan-1%

Composition C088-160419-08:

SkQ1-50 μM
agar-agar 0.25%
Sodium lactate-5%
Glycerin-10%
aloe juice-10%
algae extract (Seafill)-2%
beta-gluucan-1%
Easyliance-2%
SLMW hyaluronate-0.05
Biotin-100 μM

Results of studying the stability of SkQ1 at +60 C (SkQ1 concentrations are shown in μg/ml):

| Time at | Composition | Composition |
|---|---|---|
| +60° C., d | C088-160419-07 | C088-160419-08 |
| 0 | 26.74 | 28.52 |
| 4 | 28.05 | 29.91 |
| 8 | 26.47 | 22.20 |
| 11 | 28.88 | 19.69 |
| 15 | 25.94 | 13.22 |
| 18 | 25.94 | 11.15 |
| 22 | 17.40 | 9.05 |
| 25 | 14.46 | 7.93 |
| 29 | 10.20 | 6.94 |
| 32 | 8.75 | 4.43 |

Predicted degrees of degradation:

| No of | −k′ d − 1 | Degradation rate for 365 days of storage, % | | | | |
|---|---|---|---|---|---|---|
| composition | 60 C. | 60 C. | 25 C. | 8 C. | 4 C. | 2 C. |
| C088- | −0.03693 | 100.0% | 68.7% | 30.1% | 23.7% | 21.0% |
| 160419-07 | | | | | | |
| C088- | −0.05487 | 100.0% | 82.2% | 41.2% | 33.1% | 29.6% |
| 160419-08 | | | | | | |

Stability at +37 C was studied for the composition C088-160419-07:

Sample preparation: 1 V of the sample+1 V 0.5 M of sodium bromide, heat for 15 minutes while mixing, +9 V 96% ethanol, mix, centrifuge. To 500 ml of super add 500 ml of 0.1 M phosphoric acid in water, centrifuge, transfer to vials. The final dilution of 22 times. HPLC sample volume is 100 μl, detection at 260 nm.

### Results:

| | Storage 37 C., | C., |
|---|---|---|
| No of sample | 24 hours | μg/ml |
| C088-160419-07-37-0 | 0 | 29.77 |
| C088-160419-07-37-1 | 14 | 27.88 |
| C088-160419-07-37-2 | 28 | 27.22 |
| C088-160419-07-37-3 | 42 | 26.44 |
| C088-160419-07-37-4 | 56 | 24.71 |
| C088-160419-07-37-5 | 70 | 24.58 |
| C088-160419-07-37-6 | 84 | 24.56 |
| C088-160419-07-37-7 | 98 | 24.05 |
| C088-160419-07-37-8 | 112 | 25.29 |
| C088-160419-07-37-9 | 126 | 22.89 |

Based on the above data, you the following stability was predicted for the composition during long-term storage:

| No of | −k′ d − 1 | Degradation rate for 365 days of storage, % | | | | |
|---|---|---|---|---|---|---|
| composition | 37 C. | 37 C. | 25 C. | 8 C. | 4 C. | 2 C. |
| C088- | −0.00169 | 45.1% | 23.0% | 7.7% | 5.9% | 5.2% |
| 160419-07 | | | | | | |

Conclusion: the composition stability is acceptable, especially for storage at 2-8 C. The result may improve after analyzing the data at 25 C.

Also, in our experiments, we obtained data demonstrating acceptable stability of SkQ1 in compositions that include polyvinyl alcohol or the Aquaxyl.

1. A composition containing
(i) a mitochonrially targeted antioxidant of general formula 1: 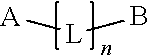
wherein:
A is a quinone antioxidant having a following structure: 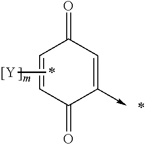
and/or reduced (quinole) form thereof, wherein:
m is an integer from 1 to 3:
Y is lower alkyl or lower alkoxy
L is a linker group, comprising straight or branched hydrocarbon chain which can be optionally substituted by one or more substituents and optionally contains one or more double or triple bonds; and
n is integer from 1 to 40;
B is a targeting group comprising SkZ, wherein Sk is a lipophilic cation; and Z is a pharmacologically acceptable anion.
(ii) and a polymer, wherein the polymer is selected from polyacrylate (carboxyvynil polymer), agarose, agar-agar or hyaluronic acid.

2. A composition according to claim 1 wherein the mitochonrially targeted antioxidant is SkQ1.

3. A composition of the claim 2 of the following content:

| | |
|---|---|
| Isopentyldiol | 5% |
| Aquaxyl (xylitylglucoside and anhydroxylytol and xylitol) | −3% |
| Propyleneglycol | 3% |
| Sodium polyacrylate | 0.9% |
| Ethylhexylglycerol | 0.3% |
| Lactic acid | 0.2% |
| SkQ1 − 6 ug/ml. | |

4. Method of stabilization of SkQ1 in a pharmaceutical or cosmetic composition by addition of lactic acid into such composition

5. Stabilized pharmaceutical or cosmetic composition of SkQ1 containing lactic acid.
